(19) United States
(12) Patent Application Publication
(10) Pub. No.: US 2005/0008608 A1
(43) Pub. Date: Jan. 13, 2005

(54) Odor-mitigating compositions

(76) Inventors: Stephen L. Parkhurst, Cedar Park, TX (US); Morey E. Osborn, Muskogee, OK (US)

Correspondence Address: M. Michelle Muller;Vinson & Elkins L.L.P., 2300 First City Tower, 1001 Fannin Street, Houston, TX, 77002-6760, US

(21) Appl. No.: 10/614,417

(22) Filed: Jul. 7, 2003

(51) Int. Cl. A61L 9/015; A61L 9/02

(52) U.S. Cl. 424/76.2

(57) ABSTRACT

The invention includes compositions, devices, systems and methods for mitigating odors, pollutants and toxins from a gaseous or liquid environment. The present invention relates to devices that contain a formulation for removing foul odors from the air. In certain embodiments the present invention provides odor-mitigating reagents that are substantially incapable of leaching from the device during ordinary use. The present invention also contemplates applications where the invention can be safely worn in contact with the user's skin or hair. Certain embodiments of the invention therefore particularly relate to devices such as diapers, shoe liners, sanitary pads, wound dressings, face masks and the like. Another aspect of the invention contemplates applications wherein the device can be used in indoor and outdoor settings where it is desirable that the odor-controlling reagents not leach out of the device as a result of contact with liquids. Related embodiments of the invention include landfill odor abatement covers and various interior and industrial air fresheners.

### BACKGROUND OF THE INVENTION

1. Field of the Invention

The present invention relates to the field of compositions devices, systems and methods for removal of undesired odors, pollutants and toxins.

2. Description of Related Art

Since mankind first became aware of the importance of hygiene, the use of antiseptics, sterilization techniques and other similar cares, the issue of malodors, and their potential adverse effects on health and quality of life have been a concern. Bad odors are sometimes an indicator of danger, such as in the case of decay where they can alert one to the possibility of infection or lurking disease. As often as not however, repugnant odors are little more than an unpleasant experience. As such they may not always be indicative of a potential health risk, but rather negatively effect quality of life. Thus, for both nuisance and health reasons, methods have been sought to eliminate or substantially alleviate bad odor experiences wherever they are encountered.

To that end, many devices and techniques have evolved through time to cope with the malodor experience. Such methods or techniques have included masking odors with perfumes, displacing odors with fans or blowers, using activated carbon as malodor absorbers, using electrostatic precipitators, candles, incense and the like. These methods and devices, though somewhat effective in lessening the impact of malodors, do not actually eliminate the malodorous substances themselves.

For example, in the case of activated carbon, malodorants are not changed and may in fact be desorbed as a result of temperature fluctuations or interior carbon particle saturation-thus rendering the method less than optimally effective. This is because the mechanism involved, which entails three separate processes (condensation, Van der Waals attraction and capillary action to the carbon particle interior), leaves the malodorous substances intact. Scrutiny of other traditional methods reveals they too leave the undesired substances intact. In electrostatic precipitation for instance, one sees a process that is fundamentally a flocculation of charged dust particles that are collected at a second stage. However, odor removal with this technique requires adsorption of noxious matter onto the targeted dust particles. This approach is clearly limited by the volatility and adsorbing propensity of the molecules involved. In any case, the odoriferous materials are not converted to less offensive compounds. Similarly, displacement and perfume masking techniques also leave the offending substances unchanged.

Recently, a fundamentally new approach to odor control has been described by the present inventors in U.S. Pat. No. 6,528,014. This “head on” approach uses chemical conversion as the mechanism by which the malodors are rendered innocuous. Certain aspects of the technology described in this patent contrast to the traditional techniques for odor control described above, as it substantially eliminates malodorous substances to benign materials by chemical conversion of the noxious particles or molecules themselves. Additionally, the technology described in U.S. Pat. No. 6,528,014 is shown to be enhanced by the use of substances called “promoters.” Appropriate promoters were described as being water-soluble ethylene oxide or propylene oxide derivatives, or mixtures of such.

While the technology of U.S. Pat. No. 6,528,014 is superior over its prior art, it is not applied as easily as would be desirable in all circumstances, for use in mitigating odors or other contaminants, from liquids, or under conditions where leaching of the reagents may be of concern. Thus, there remains a need for improved compositions, systems and methods that efficiently remove, in whole or in part, odors or other contaminants from liquids, surfaces and air, and in situations where leaching of reagents is undesirable.

### 3. BRIEF SUMMARY OF THE INVENTION

This present invention includes various embodiments of a composition comprising an odor-mitigating reagent (OMR) contacted on or within a solid support. The OMR reacts with malodorants or other contaminants to remove them, in whole or in part, from a surrounding environment. In certain embodiments, the present invention can also include compounds known as promoters to improve the rate of chemical conversion of malodors or other contaminants by the OMR.

In certain embodiments, the disclosed composition comprises an OMR that has one or more functional groups capable of chemically neutralizing one or more of the target malodorous or other contaminant particles. Thus, in certain embodiments the OMRs of the present invention comprise one or more functional groups acting as a Lewis Acid, Lewis Base, oxidizing agent, reducing agent, or any other functional groups that will chemically neutralize the target particles. Alternatively, in certain embodiments, the OMR of the present invention may be non-polymeric or polymeric. In certain embodiments, use of a polymeric OMR is preferred to provide anti-leaching or other desirable qualities to the composition. In other embodiments, the presently disclosed OMRs may be used alone, or in any combination with each other, to mitigate the presence of unwanted particles. One of skill in the art can readily determine which OMR or combination of OMRs would be useful under various conditions and in light of the particular undesired target particle or particles.

Optionally, in other embodiments, the present composition comprises, in addition to the OMR, one or more promoters. The promoter can be non-polymeric, polymeric or a mixture thereof. In certain embodiments, the promoter can provide desirable qualities to the composition; for example, in certain embodiments a polymeric promoter can serve to render the present composition non-leaching or to provide other desirable qualities.

In certain embodiments, the presently disclosed compositions can further comprise additional components that confer characteristics to the composition as would be desired by one skilled in the art, or as desired by the user of the presently disclosed compositions. For example, one can include a dispersant, antibiotic, anti-fungal, or other disinfectant, perfume or other fragrance, polishing agent, or other desired additional component.

The presently disclosed compositions are contained in or on a solid support. The solid support can be any material that serves to allow the OMR to chemically interact with and remove, in whole or in part, the target particles. Exemplary useful solid supports are listed below. The solid support useful according to the present disclosure can be contacted with the disclosed compositions by any method known to those of skill in the art. For example, without limitation, the solid support can be configured around the composition, can be impregnated with the composition, the composition can be adsorbed onto the solid support, or the composition components can be chemically linked or otherwise bound to the solid support.

The presently disclosed compositions are amenable to a variety of useful applications as will be readily apparent to one of skill in the art. Included among these are devices that incorporate the disclosed compositions and are intended for use in contact with the skin, hair or clothing of a user, especially a human user or are configured so that leaching or migration of reagents or promoters is minimized. Such embodiments include, but are not limited to, devices such as disposable diapers, foot-wear liners, wound dressings, sanitary pads, tampons, incontinence pads, flatulence pads, hair caps or covers, face masks, deodorant pads, clothing, and footwear. Alternative embodiments include for example but without limitation, landfill abatement covers, fabric, carpet, cabin air filters, other air filters such as high efficiency particulate air (HEPA) filters, forced-air and ambient-air air fresheners, fume extractor, food packaging, pet litter, bags and containers for decomposing organic matter, animal waste or other waste, vacuum bags, and in medical applications such as colostomy bags, urine bags, pet litter, body bags, bed pans and other containers for human waste or biological material. Still further embodiments include applications wherein the odor-mitigating device operates through exposure to sources of unregulated or forced air, and embodiments wherein the odor-mitigating device operates through contact with contaminant-bearing liquids.

In further embodiments, the devices of the present invention can also contain layered or segregated composites, wherein one or more of the composites comprises an OMR having a functional group capable of acting as a Lewis Acid, and one or more composites further comprise an OMR having a functional group capable of acting as a Lewis Base, or alternately, where one or more of the composites comprises an OMR having a functional group capable of acting as an oxidizing agent, and one or more composites further comprise an OMR having a functional group capable of acting as a reducing agent.

In other embodiments, the present disclosure contemplates any method of using, incorporating into an additional system or otherwise employing the disclosed compositions, devices or applications.

### 4. DETAILED DESCRIPTION OF THE INVENTION

This invention comprises compositions, devices, systems and methods for using one or more odor-mitigating reagents (OMRs) that react with malodorants or other contaminants to remove them, in whole or in part, from a surrounding environment. The invention can also include promoters to improve the rate of chemical conversion or neutralization of malodors or other contaminants by the OMRs.

Odor-mitigating compositions, as described, herein, are compositions that are effective to partially or entirely remove from the air, from liquids or from surfaces, odors, contaminants or other undesired matter. While in preferred embodiments, the odor-mitigating compositions are directed to mitigation of malodorous compounds, the odor-mitigating compositions of the present invention will neutralize any contaminant that will chemically react with the OMRs. Thus the presently disclosed odor-mitigating compositions are effective to mitigate the presence of contaminants such as malodorants, pollutants, toxins, irritants, microorganisms (such as bacteria, yeast or fungi), viruses and the like.

In general, malodorous materials can be classified into three general chemical types. They are Lewis bases, such as indoles or ammonia, which are encountered in restrooms or hair salons, predominantly neutral compounds such as hydrogen sulfide or mercaptans experienced mostly in bathrooms and Lewis Acids such as butyric acid found in disposable diapers or baby spit-up. In each case there is functionality or a “chemical handle,” which can be exploited chemically to eradicate the odor causing substance. In the case of mercaptans, for example, even mild oxidizers can convert them to sulfites, sulfates or other non-volatile, and non-odorous oxidized derivatives. Or, in the case of ammonia, a high molecular weight carboxylic acid can be employed to convert it to a non-volatile salt so it can no longer be detected in the air by primary or secondary human neural receptor sites in the nasal or oral passages. Similarly, other unwanted matter will also have a chemical functional group, or other activity, that can be chemically altered or disabled by one or more of the OMRs of the present invention to remove or neutralize, in whole or in part, that unwanted matter.

“Malodorants” or “malodors” include any objectionable odor-causing chemical agent. “Contaminants” refer to malodorants, pollutants, toxins, irritants, viruses and the like, and microorganisms, such as bacteria, yeast or fungi, wherein said contaminants are capable of reacting with the OMRs of the invention and thus being partially or completely removed from the surrounding or target environment. The term contaminant therefore includes chemical compounds that will react with the OMRs of the present invention (including toxins such as carbon monoxide). While in many portions of the present disclosure the invention is discussed in terms of compositions, devices, systems or methods for mitigating malodorants or malodors, the invention applies equally well to remove any other contaminant that can chemically react with a functional group of one or more OMRs, as taught herein. Accordingly, applications of the presently disclosed compositions, devices, systems and methods to remove contaminants is specifically contemplated herein.

Malodorant or contaminant particles that can be mitigated by the present invention are individual molecules such as ammonia, mercaptans, indoles, pyrroles, hydrogen sulfide, carboxylic acids (e.g. butyric acid), carbon monoxide and the like. Malodorant or contaminant particles that can be mitigated by the present invention can also be larger aggregates of molecules such as bacteria, fungi, spores, viruses and the like.

1. Odor-Mitigating Reagents

The present invention provides OMRs that are capable of reacting with malodorants and other contaminants and sequestering them from their surrounding environment. In certain preferred embodiments, therefore, the presently disclosed compositions comprise one or more OMRs with a functional group such as a Lewis Acid, Lewis Base, oxidizing group (such as a sulfur-labile functional group), a reducing group, or other functional group. OMRs that contain functional groups that are Lewis Bases will react with compounds that are Lewis Acids to produce their conjugate salt. The result is that the malodorant becomes permanently bound to the OMR, and is thereby partly or completely rendered neutral or undetectable by the user. Conversely, OMRs that contain functional groups that are Lewis Acids will react with compounds that are Lewis Bases to similarly convert those compounds into a bound form that is partly or completely neutral or undetectable. In a similar manner, OMRs having oxidizing agents or hydro-sulfur labile functional groups will bind to odor-causing compounds such as mercaptans and hydrogen sulfide. The same mechanism can be used to remove contaminants that will react with an OMR containing a functional group that is a reducing agent. Thus, the principles of the present invention can be readily applied to classes of OMRs that are reducing agents, wherein the reagent can be used to remove any contaminant species that will react with the reducing agent. Other functional groups that can be used to react with malodorous or other unwanted matter will be apparent to those of skill in the art.

The present invention therefore provides OMRs that contain one or more functional groups that can act as a Lewis Base. A Lewis Base can, for example, but used to convert malodorous compounds containing carboxylic acid to their corresponding conjugate salt. Lewis Bases are defined as chemical compounds that can act as an electron pair donor. Once converted, the malodorous compound becomes bound to the reagent. Examples of non-polymeric Lewis Bases useful in the present invention include, sodium carbonate, Calcite and potassium carbonate. Other appropriate non-polymeric Lewis Bases will be apparent to those of skill in the art. Non-polymeric and polymeric compounds can have also one or more functional groups that can act as a Lewis Base and therefore are useful as Lewis Base reagents in the present invention.

The present invention also provides for OMRs that contain one or more functional groups that can act as a Lewis Acid. A Lewis Acid can, for example, convert amines into their conjugate salts. Lewis Acids are defined as chemical compounds that can act as an electron pair acceptor. Useful non-polymeric Lewis Acid reagents include ascorbic acid, aspartic acid, phenol, citric acid, maleic acid, oxalic acid and succinic acid. Other appropriate non-polymeric Lewis Acids will be readily apparent to one of skill in the art. As with their Lewis Base counterparts, non-polymeric and polymeric compounds can also have one or more functional groups that can act as a Lewis Acid and therefore act as an OMR in the present invention.

A further OMR provided by the present invention are oxidizing agents or hydro-sulfur labile compounds. Oxidizing agents and hydro-sulfur labile compound are especially useful for converting sulfurous compounds such as hydrogen sulfide and mercaptans into non-volatile, non-odorous compounds. Because these compounds are strongly malodorous even at very low levels, chemical capture of these compounds by the OMRs of the present invention is a highly effective means of odor control in many situations. In certain preferred embodiments the oxidizing agent is a chlorine donor or bleach such as the sodium dichloroisocyanurate, disodium chloroisocyanurate, trichloroisocyanuric acid, dipotassium chloroisocyanurate, dilithium chloroisocyanurate, sodium hypochlorite, calcium hypochlorite, potassium hypochlorite, magnesium hypochlorite, lithium hypochlorite, hypochlorous acid and the like, but other common and exotic oxidizers that can be used in the present invention are listed in U.S. Pat. No. 6,528,014, herein incorporated by reference, and in Tables 1-3.

In a preferred embodiment, appropriate oxidizing agents can be used for the neutralization of carbon monoxide. Such embodiments would include industrial air filtration systems, face masks and the like.

TABLE 1
Common oxidizers useful for conversion of hydrogen
sulfide.

| | |
|---|---|
| 1. | Sodium dichloroisocyanuric acid. |
| 2. | Potassium dichloroisocyanuric acid. |
| 3. | Sodium trichloroisocyanuric. |
| 4. | Potassium trichloroisocyanuric. |
| 5. | Potassium permanganate. |
| 6. | Sodium permanganate. |
| 7. | Sodium hypochlorite in wet solvent. |
| 8. | Potassium hypochlorite in wet solvent. |
| 9. | Hypochlorous acid in wet solvent. |
| 10. | Calcium oxide. |
| 11. | Magnesium oxide. |
| 12. | Chromium trioxide. |
| 13. | Manganese dioxide. |
| 14. | Chromium trioxide-pyidine complex. |
| 15. | Lead tetraacetate. |
| 16. | Barium oxide. |
| 17. | Cadmium oxide. |
| 18. | Lead oxide. |
| 19. | Strontium oxide. |
| 20. | Mercury oxide. |
| 21. | Chromic acid. |
| 22. | Hydrogen peroxide - wet. |
| 23. | Sodium peroxydicarbonate. |
| 24. | Potassium peroxydicarbonate. |
| 25. | Metachloroperbenzoic acid. |
| 26. | Trifluroperacetic acid. |
| 27. | Trichloroperacetic acid. |
| 28. | Perbenzoic acid. |
| 29. | Potassium chromate. |
| 30. | Sodium chromate. |
| 31. | Sodium metaperiodate. |
| 32. | Potassium metaperiodate. |
| 33. | Copper oxide. |
| 34. | Cobalt oxide. |
| 35. | Osmium tetroxide. |
| 36. | Titanium dioxide. |
| 37. | Tungsten oxide. |
| 38. | Diatomic halogens (iodine, chlorine, |
| | bromine and fluorine). |

TABLE 2
Exotic oxidizers useful in this invention

| CHEMICAL | GAS # | CROSS-REF |
|---|---|---|
| tert-BUTYLPEROXY | [26748-41-4] | |
| NEODECANOATE | | |
| tert-BUTYL | [26748-38-9] | |
| PEROXYNEOHEPTANOATE | | |
| tert-BUTYLPEROXY | [13467-82-8] | tert-BUTYL PEROCTOATE |
| OCTOATE | | |
| tert-BUTYL | [0-0-0] | |
| PEROXYPHENOXYACETATE | | |
| 3-tert-BUTYLPEROXY 3- | [25251-51-81 | |
| PHENYLPHTHALIDE | | |
| tert-BUTYLPEROXY | [927-7-1] | |
| PIVALATE | | |
| tert-BUTYLPEROXY iso- | [2372-21-6] | |
| PROPYL CARBONATE | | |
| tert-BUTYLPEROXY | [0-0-0] | |
| STEARYL CARBONATE | | |
| tert-BUTYLPEROXY o- | [22313-62-8] | |
| TOLUATE | | |
| tert-BUTYLPEROXY-3,5,5- | [13122-18-4] | |
| TRIMETHYLHEXANOATE | | |
| O,O-tert-BUTYL-O-iso- | [0-0-0] | |
| PROPYL PEROXYCARBONATE | | |
| CHLOROPEROXYBENZOIC | [0-0-0] | |
| ACID | | |
| 3- | [937-14-4] | m-CHLOROPERBENZOIC |
| CHLOROPEROXYBENZOIC ACID | | ACID |
| 9(11),22-CHOLESTADIEN-246- | [0-0-0] | |
| METHYL-5,8-PEROXY-3b-OL | | |
| ACETATE | | |
| 6,9(11),22- | [0-0-0] | |
| CHOLESTATRIEN-246-METHYL-5,8- | | |
| PEROXY 36-OL ACETATE | | |
| 6,9,(11),22- | [0-0-01 | |
| CHOLESTATRIEN-246-METHYL-5,8- | | |
| PEROXY 3b- | | |
| OL ACETATE | | |
| COPPER (II) OXYACETATE | [52503-63-6] | |
| COPPER OXYCHLORIDE | [1332-40-7] | |
| COPPER (II) OXYCHLORIDE | [1332-40-7] | COPPER OXYCHLORIDE |
| COPPER OXYCHLORIDE | [0-0-0] | |
| SULFATE | | |
| TRIMETHYLCYCLOHEXANE | [6731-36-8] | 1,1-BIS(tert-BUTYLPEROXY)-3,3,5- |
| | | TRIMETHYLCYCLOHEXANE |
| DICETYL PEROXY | [26322-14-5] | |
| DICARBONATE | | |
| DICYCLOHEXYL PEROXY | [1561-49-5] | |
| DICARBONATE | | |
| DI-2-ETHOXYETHYL | [52373-74-7] | |
| PEROXYDICARBONATE | | |
| DI(2- | [16111-62-9] | |
| ETHYLHEXYL)PEROXYDICARBONATE | | |
| 2,5-DIHYDROPEROXY-2,5- | [3025-88-5] | |
| DIMETHYLHEXANE | | |
| 2,4-DIHYDROXY-2-METHYL | [0-0-0] | |
| 4-HYDROPEROXYPENTANE | | |
| DIMETHOXY iso- | [0-0-0] | |
| PROPYLPEROXYDICARBONATE | | |
| 2,5-DIMETHYL-2,5-BIS(tert- | [78-63 7] | 2,5-DIMETHYL-2,5-DI(tert- |
| BUTYLPEROXY)HEXANE | | BUTYLPEROXY)HEXANE |
| 2,5-DIMETHYL-2,5-BIS(tert- | [0-0-0] | |
| BUTYLPEROXY)HEX-3-YNE | | |
| 2,5-DIMETHYL-2,5-BIS(2- | [13052-9-0] | 2,5-BIS(2- |
| ETHYLHEXOYLPEROXY)HEXANE | | ETHYLHEXANOYLPEROXY-2,5- |
| | | DIMETHYLHEXANE |
| 2,5-DIMETHYL-2,5- | [2618-77-1] | |
| DI(BENZOYLPEROXY)HEXANE | | |
| 2,5-DIMETHYL-2,5-DI(tert- | [78-63-7] | |
| BUTYLPEROXY)HEXANE | | |
| 2,5-DIMETHYL-2,5-Di(tert- | [1068-27-5] | 2,5-DIMETHYL-2,5-Di(tert- |
| BUTYLPEROXY)-3-HEXYNE | | BUTYLPEROXY)HEXYNE-3 |
| 2,5-DIMETHYL-2,5-DI(tert- | [0-0-0] | |
| BUTYLPEROXY)HEXYNE | | |
| 2,5-DIMETHYL-2,5-DI(tert- | [1068-27-5] | |
| BUTYLPEROXY)HEXYNE-3 | | |
| 2,5-DIMETHYL-2,5-DI(2- | [13052-9-0] | 2,5-BIS(2- |
| ETHYLHEXANOYL PEROXY) | | ETHYLHEXANOYLPEROXY 2,5- |
| HEXANE | | DIMETHYLHEXANE |
| DI-(3-METHYL-3-METHOXY | [0-0-0] | |
| BUTYL)PEROXYDICARBONATE | | |
| DIMYRISTYL | [53220-22-7] | |
| PEROXYDICARBONATE | | |
| DI(PROPYL)PEROXYDICARBONATE | [16066-38-9] | |
| Di-iso-PROPYL | [705-64.6] | |
| PEROXYDICARBONATE | | |
| ETHYL-3,3-BIS(tert- | [55794-20-2] | ETHYL 3,3-DI(tert- |
| BUTYLPEROXY)BUTYRATE | | BUTYLPEROXY)BUTYRATE |
| ETHYL 3,3-DI(tert- | [55794-20-2] | |
| BUTYLPEROXY)BUTYRATE | | |
| 2-ETHYLHEXYL | [1611-62-9] | |
| PEROXYDICARBONATE | | |
| HYDROGEN PEROXY- | [0-0-01] | |
| SULFURIC UREA | | |
| 1-[(1- | [78-18-21 | |
| HYDROPEROXYCYCLOHEXYL)DIOXY] | | |
| CYCLOHEXANOL | | |
| 1- | [1226258-7] | CYCLOHEXANONE |
| HYDROPEROXYCYCLOHEXYL-1- | | PEROXIDE |
| HYDROXY CYCLOHEXYL PEROXIDE | | |
| 13(S)- | [33964-75-9] | |
| HYDROPEROXYOCTADECA-9Z,11E- | | |
| DIENOIC ACID | | |
| 13(8)-HYDROPEROXY- | [0-0-0] | |
| 9Z,11E,15Z-OCTADECATRIENOIC | | |
| ACID | | |
| MAGNESIUM | [84665-66-7] | |
| MONOPEROXYPHTHALATE | | |
| MAGNESIUM | [78948-87-5] | |
| PEROXYPHTHALATE | | |
| MAGNESIUM | [0-0-0] | |
| PEROXYPHTHALATE | | |
| HEXAHYDRATE | | |
| MONOPEROXYPHTHALIC | [78948-87-51 | |
| ACID, MAGNESIUM SALT | | |
| MONOPEROXYPHTHALIC | [84665-66-7] | |
| ACID MAGNESIUM SALT | | |
| HEXAHYDRATE | | |
| p-NITROPEROXYBENZOIC | [943-39-5] | 4-NITROPERBENZOIC ACID |
| ACID | | |
| OXOPEROXYMOLYBDENUM | [23319-63-3] | OXOPIPEROXYMOLYBDENUM |
| (PYRIDINE)HEXAMETHYLPHOSPHORAMIDE | | (PYRIDINE)HEXAMETHYLPHOSPHORAMIDE |
| OXOPIPEROXYMOLYBDENUM | [23319-63-3] | |
| (PYRIDINE)HEXAMETHYLPHOSPHORAMIDE | | |
| PEROXYACETIC ACID | [79-21-0] | PERACETIC ACID |
| PEROXYDICARBONATE | [34099-48-4] | |
| PEROXYDISULFURYL | [13709-32-5] | |
| FLUORIDE | | |
| PEROXYDOL | [0-0-0] | |
| PEROXYKETAL, CYCLIC | [0-0-0] | |
| PEROXYMONOSULFURIC | [0-0-01 | |
| ACID | | |
| PEROXYNITRITE | [14042-1-4] | |
| PEROXYNITRITE, | [157167-78-7] | |
| TETRAMETHYLAMMONIUM SALT | | |
| POTASSIUM | [15593-49-4] | |
| PEROXYDIPHOSPHATE | | |
| POTASSIUM | [7727-21-1] | POTASSIUM PERSULFATE |
| PEROXYDISULFATE | | |
| POTASSIUM PEROXY | [0-0-0] | |
| MONOSULFATE | | |
| iso-PROPYL-sec- | [0-0-0] | |
| BUTYLPEROXYDICARBONATE | | |
| SODIUM CARBONATE | [0-0-0] | |
| PEROXYHYDRATE | | |
| SODIUM | [4452-58-8] | |
| PEROXYCARBONATE | | |
| SODIUM | [7775-27-1] | SODIUM PERSULFATE |
| PEROXYDISULFATE | | |
| TETRAKIS(PYRIDINE)SILVER | [15810-50-1] | |
| (II) PEROXYDISULFATE | | |
| TETRAPOTASSIUM | [0-0-0] | |
| PEROXYDIPHOSPHATE | | |
| 2,4,4-TRIMETHYLPENTYL-2- | [0-0-0] | |
| PEROXYNEODECANOATE | | |
| 2,4,4-TRIMETHYLPENTYL-2- | [0-0-0] | |
| PEROXYPHENOXYACETATE | [15188-9-7] | |
| VINYL TRIS(tert-) | | |
| BUTYLPEROXY)SILANE | | |

TABLE 3
Hydro-sulfur labile converter functional groups

| | |
|---|---|
| 1. | Carboxylic acid anhydrides |
| 2. | Olefins |
| 3. | Alkynes |
| 4. | Carboxylic acid esters |
| 5. | Aldehydes |
| 6. | Isonitriles |
| 7. | Alkyl halides |
| 8. | Alpha diketones |
| 9. | Acyl halides |
| 10. | Diazo ketones |
| 11. | Epoxides |
| 12. | Isocyanates |
| 13. | Isothiocyanates |
| 14. | Thiocyanates |
| 15. | Vinyl ethers |
| 16. | Diazonium salts |
| 17. | Alpha-beta unsaturated carbonyls |
| 18. | Ketones |
| 19. | Alpha-beta unsaturated nitriles |
| 20. | Metal hydrides |
| 21. | Carbamates |

Non-polymeric and polymeric compounds having one or more functional groups capable of acting as an oxidizing agent are also provided by the present invention.

In certain embodiments, a reducing agent can function as an OMR. For example, reducing agents such as sodium sulfite can be used to control odors associated with urine. In a preferred embodiment, a reducing agent is incorporated into face masks employed around chlorine spills.

What constitutes an effective amount of the OMR will vary according to the application and according to the number and type of reactive groups present on the OMRs in the device that are available to interact with malodorous or otherwise undesirable compounds in the surrounding environment. Methods of determining the amount of reagent required for an intended purpose will be readily apparent to those of skill in the art.

Due to their incompatibility, it is generally not desirable to mix OMRs that contain Lewis Acids and Lewis Bases. The mixture of these compounds with one another will result in the formation of the conjugate salt of the compounds, thereby effectively reducing or eliminating the effectiveness of the OMR. Similarly, direct mixture of significant quantities of oxidizing reagent with reducing agent will also result in an undesirable reduction in the effectiveness of the OMR. In some embodiments, however, it may be desirable for relatively small quantities of an incompatible compound to be mixed with an excess amount of OMR. This procedure is acceptable as long as a sufficient quantity of unreacted functional groups remain present to chemically react with the contaminants that are to be removed from an environment.

Also, in some instances, it is desirable to use an OMR of higher molecular weight. Depending on their molecular weight, the OMRs of the present invention are classified as polymeric or non-polymeric.

“Polymeric OMR” for the purposes of the present invention means compounds capable of reacting with malodorants, or other contaminants. A polymeric OMR is a compound having a molecular weight higher than about 400 and which produces the desired anti-leaching properties. Polymeric OMRs as defined under the present invention are compounds which produce the desired anti-leaching properties. These properties are more pronounced in compounds having higher molecular weights. In general, for purpose of the present invention, polymeric OMRs are compounds having a molecular weight higher than about 400. Other exemplary polymeric OMRs will have molecular weights of about 450 molecular weight or greater, about 500 molecular weight or greater, about 600 molecular weight or greater, about 700 molecular weight or greater, about 800 molecular weight or greater, about 900 molecular weight or greater, about 1000 molecular weight or greater, about 1100 molecular weight or greater, about 1200 molecular weight or greater or about 1500 molecular weight or greater.

“Non-polymeric OMR” for the purposes of the present invention means compounds capable of reacting with malodorants or other contaminants that have a molecular weight lower than about 400. Exemplary non-polymeric OMRs will have molecular weights of about less than about 450 molecular weight, less than about 500 molecular weight, less than about 600 molecular weight, less than about 700 molecular weight, less than about 800 molecular weight, less than about 900 molecular weight, less than about 1000 molecular weight, less than about 1100 molecular weight, less than about 1200 molecular weight, and less than about 1500 molecular weight.

Polymeric OMRs have been found to be particularly useful in the present invention. Polymeric OMRs can be used as the sole OMR of the present invention, or can be combined with other compounds to enhance the efficacy of the device. In particular, when mixed with appropriate amounts of compounds such as non-polymeric OMRs, promoters, and other similar compounds, polymeric OMRs act to render these compounds substantially non-leaching or otherwise incapable of migrating from the odor-mitigating device in significant quantities.

Preparation of polymeric OMRs can be achieved by any means available to one of skill in the art. For example, polymers containing carboxylic acid and other functional groups capable of reacting with malodorous compounds or other contaminants or otherwise altered to various degrees, are readily commercially available, e.g. polyacrylates for superabsorbent applications and the like. These polymers can be modified, by adding a functional group to them, to mitigate members of the various malodorant categories described above. Such chemical modifications permit the effective substitution of the polymeric reagents for their non-polymeric counterparts. Accordingly, non-polymeric OMRs can also be covalently bound to polymers to achieve similar advantages, including to minimize leaching of the OMR from the composition. Furthermore, the polymer structure itself could act as a substitute for the support component, in essence acting both as an OMR and as a solid support for the components of the present invention.

To produce polymeric OMRs, polymers can be selected from a list of polymers having functional groups along the polymer backbone belonging to one or more of the following classes—
1. Carboxylic acids.
2. Carboxyl amides.
3. Nitriles.
4. Carboxylic acid salts.
5. Alcohols (hydroxyls).
6. Halides.
7. Olefins.
8. Carboxylic acid esters.
9. Arenes.
10. Substituted arenes.
11. Amines.
12. Ammonium salts.
13. Sulfonic acids.
14. Sulfonic acid salts.
15. Sulfuric acid.
16. Sulfate salts.
17. Mercaptans.
18. Carbamates.
19. Ketones.
20. Aldehydes.
21. Acyl halides.
22. Orthoethers.
23. Phosphoranes.
24. Phosphates.
25. Acroleins.
26. Styrenes.
27. Amino acids.
28. Alkaloids.
29. Aminoaldehydes.
30. Halohydrins.
31. Epoxides.
32. Lactones
33. Metal carbonyls, and
34. Metal Hydrides.

In preferred embodiments the functional groups will include the carboxylate salts, carboxylic acids, carboxylamides, nitrites, carboxylic acid esters (partially or fully hydrolyzed). However, alcohols, amides, amines, sulfonamides, carbamates, aldehydes, halides, phosphates, phosphate esters and ammonium salts and the like can also be used as functional groups.

Commercial polymers having important functional groups are readily available. ALCOTEX^{R}, for example, which is a partially hydrolyzed polyvinyl acetate commonly used for suspension polymerization, can be purchased in various hydrolyzed and/or neutralized states. Polyacrylates, which contain acid residues on the polymer backbone are also available. Examples of these products include FAVOR^{R} SXM 7500 from Stockhausen, and HYSORB^{R} 8200 from BASF or other superabsorbent polymers. In addition, specialty copolymers having neutralized or unneutralized functional groups are also available. Examples of these materials include CARBOPOL ULTREX^{R} 10 (polypropenoic acid) from B.F. Goodrich or A-140 (polyacrylate graft copolymer on carbohydrate) from Grain Processing Corporation.

The backbone of the polymer chain of the present invention can consist of any linear chain or branched chain polymer having the proper characteristics for effective use in the desired embodiment. Appropriate polymers include polymers, polymer blends, co-polymers, and block copolymers, as well as polyurethane, polyacrylates and polyacrylate containing polymers. In preferred embodiments, the polyacrylate polymers may contain polyacrylic acids at various levels of neutralization. Examples of appropriate polymers for use in any particular embodiment will be readily apparent to those of skill in the art.

2. Solid Support

As an additional step, the reagents and mixtures of the present invention are applied to a variety of solid supports, including powders, fabrics, meshes, webs and screens. The compositions can be contacted on, incorporated within or otherwise associated with the solid support. Non-limiting examples of solid supports useful in the present invention include polymers including the polymeric OMRs and polymeric promoters themselves, as well as extraneous polymers that do not act as an OMR or promoter, silica (fumed or precipitated), zeolite (natural and synthetic), alumina, woven synthetic fabrics including nylon and polyester fabrics, non-woven synthetic fabrics such as polypropylene or polyethylene fabrics, plastic or metal meshes, webs or screens.

A “device” in the present invention is an object that serves as a means of containment for one or more OMRs and, if applicable, the promoters and other compounds provided by the present invention. This means of containment is disposed in a manner such that the OMR is capable of reacting with environmental malodorants or contaminants. In certain embodiments a device is created by the solid support itself.

3. Promoters

Another element of certain embodiments of the present invention are compounds called “promoters.” Promoters are compounds that are semi-liquid and humectant. They can be either non-polymeric or polymeric compounds.

“Polymeric promoter” for the purposes of the present invention means humectant compounds that, in the presence of OMRs, promote the chemical conversion of malodorants or other contaminants. Polymeric promoters as defined under the present invention are compounds which produce the desired anti-leaching properties. These properties are more pronounced in compounds having higher molecular weights. In general, for purpose of the present invention, polymeric promoters are compounds having a molecular weight higher than about 700. Other exemplary polymeric promoters will have molecular weights of at least about 750 molecular weight, at least about 800 molecular weight, at least about 900 molecular weight, at least about 1000 molecular weight, at least about 1200 molecular weight, and at least about 1500 molecular weight.

“Non-polymeric promoter” for the purposes of the present invention means humectant compounds that, in the presence of OMRs, promote the chemical conversion of malodorants or other contaminants, Non-polymeric promoters have a molecular weight of less than about 700. Exemplary non-polymeric promoters will have molecular weights of less than less than about 750 molecular weight, less than about 800 molecular weight, less than about 900 molecular weight, less than about 1000 molecular weight and less than about 1200 molecular weight.

By way of explanation and non limitation, the improved performance of OMRs in the presence of promoters is the result of a principle known as “solution-phase kinetics”. Since transformations occurring between a gas and solid are notoriously inefficient as compared to ones occurring in solution (owing to the requirement of effective molecular collision orientation, as well as a statistically limited number of opportunities to have an effective collision resulting in the desired chemical transformation), promoters are thought to impart a “solution-like” environment to the OMR. This “solution-like” environment imparted by the semi-liquid and humectant promoters can improve the likelihood of the desired collisions taking place between the reagent and malodorants or other contaminants. The relatively high strength of the hydrogen-bonds formed between the promoters and water is also thought to be the reason that reagent-containing, porous fabric supports used in certain forced-air embodiments of the present invention stay moist and functional for extended periods (typically several months).

The promoters of the present invention are also believed to increase malodorant conversion in embodiments of the invention where the OMR comes in direct contact with a liquid solution containing malodorants or contaminants rather than with contaminated air. In these cases, the promoter acts to decrease interfacial tension between the contaminated solution and the OMRs. The result is again to increase the rate at which the reagents chemically convert the contaminants.

Promoters of the present invention are compounds that have humectant qualities that attract or retain (or both) water. In certain embodiments the promoters of the present invention form hydrogen bonds with water having bond strength of at least about 1.0 kcal/mole, at least about 1.5 kcal/mole, at least about 2.0 kcal/mole, at least about 2.5 kcal/mole, or at least about 3.0 kcal/mole. In preferred embodiments, the promoters have hydrogen bonding strengths of 1.5 to 3.5 kcal/mole, and in highly preferred embodiments, 2.0 to 3.0 kcal/mole.

Ethylene oxide and propylene oxide derivatives are effective promoters including, for example, polyethylene glycol, polypropylene glycol, nonionic surfactants such as glucan P-20 (Amerchol) and ethoxylates such as Tergitol® 9.5 (Union Carbide), Alfonic® Nonionic (Vista Chemical Co.), JL 80×nonionic (Huntsman), cetyl-range ethoxylated nonionics, lauryl-range ethoxylated nonionic surfactants and the like. Other effective promoters include cationic surfactants, carbowax, ethoxylate and the like. Less preferred are anionic surfactants which can be used as the sole promoter or as a supplement to other promoters. Similarly, quaternary ammonium salts can also be used as promoters in this invention, owing to their humectant qualities. They have the additional attribute of acting as a disinfectants. In less preferred embodiments of the present invention, soaps can also be used as promoters. Other compounds that have the desired humectant properties will be readily apparent to those of skill in the art.

The “solution-like” environment produced by the addition of promoters is achieved by the complexing of the promoters with water, effectively forming an aqueous solution. The formation of this “solution-like” environment requires the presence of water in association with the promoters of the present invention. The water can be added during the formulation process, or can be added at some later point prior to the use of the present invention for the elimination of odors or contaminants. Alternatively, even if no water is affirmatively added to the promoters of the present invention, adventitious water will become associated with the promoter, that is, water is absorbed by the promoter from its surrounding environment. Alternately, the promoter may be added to the device in its dry form and become moistened prior to or during use. For example, polyethylene glycol (PEG) is a dry substance that can be used as a promoter. PEG may be added to a device such as a diaper in its dry form and become moistened during actual uses when contacted by urine.

Certain promoters also exhibit some solvency attributes beyond mere humectancy, which can further add to their effectiveness. In certain embodiments, such promoters can function to cross neutralize of Lewis acid and Lewis base malodorant molecules. Thus, it appears that promoters of the present invention are useful, to some degree, in fostering neutralization (thereby elimination) of odor through promotion of primary odor constituent interaction—i.e. the promoters assist the malodors in neutralizing each other rather than being neutralized any OMRs.

The presence of non-polymeric promoters (including nonionic surfactants made from multiple ethylene or propylene oxide additions to surfactant range alcohols) used as dispersion agents, has the beneficial side effect of reducing surface tension, thereby minimizing a partial barrier to absorption of malodors or contaminants). Similarly, promoters can act to facilitate the desired faster solution-phase chemistry between malodor or contaminant molecules in liquid and the odor-fighting reagents reducing interfacial tension which in turn reduces the partial barrier between the compositions of the present invention odorous or contaminated liquid.

Low molecular weight promoters can further be used as a processing aid in dispersing higher molecular weight polymers throughout the mixtures of the present invention. Low molecular weight promoters such as a low molecular weight polyether that is a non-ionic surfactant ethylene oxide adduct having 6 or more ethylene oxide units and the like are useful dispersants.

4. Additional Components of the Compositions

In certain embodiments, the present invention may additionally contain certain additives that improve the manufacturing process or lend other desirable qualities to the final product. For example, in certain embodiments it may be desirable for a disclosed composition to also contain compounds with disinfectant properties. Alternatively, it may be desirable for a composition or device to emit a pleasant scent, for instance, as an indicator that the odor-mitigating device is functioning or as a perfume or fragrance.

Other desirable additives include polishing agents (including use of reducing agents to reduce excess oxidizing agent), or compounds that can serve as disinfectants and bacteriocidal compounds. A particularly preferred reducing agent for this purpose is sodium sulfite. Examples of disinfectants that can be used in present invention include ethanol, quaternary ammonium salts, biguanides, chelators and other disinfectants and bacteriocidal compounds known to those of skill in the art. Additionally, certain OMRs such as sodium hypochlorite or sodium dichloroisocyanurate and act as both an OMR and as a disinfectant. Further desirable additives that do not substantially interfere with the function of the OMRs will be readily apparent to those of skill in the art.

5. Substantially Non-Leaching Compounds

In certain embodiments, it is desirable to include substantially non-leaching compounds into the presently disclosed compositions or devices. Such compounds can be included by virtue of using an OMR, promoter or other additive that is itself substantially non-leaching, such as, for example but without limitation, polymeric or polymer-bound OMRs or polymeric promoters described above, or by adding a separate substance or additive that is substantially non-leaching.

In the context of the present invention, “substantially non-leaching” means that a compound incorporated into a disclosed composition or device remains substantially within the composition or device, when contacted by moisture or liquids in the ordinary course of use. For example, in the context of disposable diaper embodiments of the present invention, substantially non-leaching means that in the normal course of use wherein the diaper comes in contact with waste products and/or body moisture, the substantially non-leaching compound within the diaper does not leave the device in amounts sufficient to cause skin-irritation or other ill-effects upon the user, or to an extent that the odor-mitigating properties of the diaper are substantially impaired.

6. Formulation

The composition of this invention in certain preferred embodiments consists of two basic components. They are: 1) non-polymeric or polymeric promoters (or both) and 2) polymeric OMRs, which can be augmented at some level with non-polymeric OMR. Mixtures of promoters can be prepared by mixing one or more non-polymeric promoters (such as low molecular weight nonionic surfactants) to a concentration of 0 to 100% non-polymeric promoter, preferably 0.1% to 80% non-polymeric promoter, more preferably 0.5% to 50% non-polymeric promoter, with any one or more of a number of different polymeric promoters. In many preferred embodiments the polymeric promoters used in the mixture are high molecular weight polyethers, including mixtures of polyethylene oxide, polypropylene oxide or carbowax. While the addition of lower molecular weight material is not required to practice the present invention, the presence of lower molecular weight material can serve many beneficial purposes. In particular, when the higher molecular weight material is mixed with the lower molecular weight material, the latter effectively acts as a softener, which can aid in dispersing the material in processing.

In general, an odor-fighting composition can be prepared by mixing a selected OMR in the amount of 0.01% to 100% (by weight), preferably about 2% to 100%, more preferably about 10% to about 99.9%, most preferably about 20% to about 90% with a selected promoter or mixture of promoters. In some cases, depending on the application, an appropriate amount of a non-polymeric OMR (for example, an oxidizer such AS the sodium salt of dichloroisocyanuric acid for malodorous substances) can be added as well for a more efficacious composition. The presence of polymeric reagent and/or polymeric promoter tends to severely curtail leaching of non-polymeric components as long as they are not present in excessive quantities. The polymeric/non-polymeric ratio of the composition may be anywhere from 0% to 100% polymeric, but in preferred embodiments the compositions of the present invention will be at least about 20% polymeric, preferably at least about 35% polymeric, more preferably at least about 50% polymeric, most preferably at least about 70% polymeric (by weight). In general, compositions with higher polymeric compound will tend to have better anti-leaching properties.

Sometimes, to enhance the desired Lewis Acid or Lewis Base properties of a specific OMR, acid or base pretreatment of the polymeric reagent will be required before the final composition is put together, such as prior to contacting the OMR and the solid support.

In addition to promoting conversion of contaminants by the OMRs, the presence of non-polymeric promoter can serve other functions, for example, they may act as a dispersant. It is therefore desirable in certain embodiments to use a mixture of non-polymeric and polymeric promoters. Since only small quantities of non-polymeric promoter may be required for these additional functions, the proportion of polymeric promoter generally can be greatly elevated without compromising the humectant qualities of the composition. Thus, the requisite promoter part of this invention can range from 100% polymeric to 100% non-polymeric, including at least about 20% polymeric, at least about 40% polymeric, at least about 60% polymeric or at least about 80% polymeric.

When formulating, one tends to include more polymeric promoter or other polymeric components when a more anti-leaching quality is required, and less polymer if leaching is less of an issue, or if the polymeric odor-fighting reagent portion of the composition is sufficient to reduce component migration or leaching (or both).

Due to their inherent incompatibility, Lewis Acids and Lewis Bases are generally not mixed in a single reagent package. Thus, certain embodiments of the present invention will comprise layered or segregated composites that allow both Lewis Acid and Lewis Base containing OMRs or reagent packages to act upon air or liquids containing contaminants, without permitting any significant portion of the reagents within those composites to come in direct contact with each other. For example, separate layers containing reagent or reagent package can be incorporated into a disposable diaper, such that the contents of the diaper come in contact with both layers, but the reagents within they layers remain substantially separated during use.

Alternatively, where it is not possible to prepare layered or segregated composites, the Lewis Acid and Lewis Base containing OMRs or reagent packages can be applied serially. In this case, the compositions or devices of the present invention can be formulated in pairs meant to be used in series.

A highly preferred embodiment of the present invention is the formulation of odor-mitigating compositions comprising a chlorine donor and bleach component in association with a polymer composition capable of neutralizing hydrochloric acid. These compositions can be particularly valuable in applications involving the neutralization of malodorants associated with urine. Not to be bound by theory, it is believed that when odor-mitigating devices are exposed to urine and its associated malodorants, the chlorine donor or bleach component of the device will react with the malodorant compounds, neutralizing them and producing hydrochloric acid as a byproduct of the reaction. The hydrochloric acid that is produced reacts in turn with the polymer composition and is also neutralized. Preferred polymers for use in such embodiments include super-absorbent polymers, including polyacrylate polymers, that contain carboxylate groups. In such polymers, carboxyl groups can be present either as carboxylic acid or as carboxylate salts. Preferred polymers for the present invention will tend to have a high percentage of carboxylate salt groups present, or at least a quantity of carboxylate groups that is sufficient to neutralize any hydrochloric acid that is produced by the reaction of the chlorine donor or bleach components of the device.

7. Applications

The compositions, devices and methods of the present invention can produce a significant reduction in the amount of malodorant perceived by users and significant reductions in the quantity of other contaminants found in a particular environment. While smell is highly objective, certain empirical measurements of malodor have been developed, including “panel tests,” wherein a panel of testers rate the malodorous qualities of various compositions, or gas-chromatographic “head-space” analyses. Gas-chromatographic analyses may involve gas-chromatography-mass spectral analysis (GC-Mass Spec), or may involve gas-chromatographic analysis alone based on retention time analysis. Such methods are well known to those of skill in the art. Such methods are routinely used to identify and quantify air-borne compounds by introducing a volume of contaminated air into the spectrometer. A significant reduction in malodorant or other contaminants under the present invention may comprise a reduction in malodorant or other contaminant of at least 1%, 2%, 5% 10%, 20%, 30%, 40%, 50%, 60%, 70% 80%, 90%, 95%, 98%, 99% or 100% as measured either by panel tests, by chromatographic “head-space” analysis, or by any other appropriate measurement of malodorant or contaminant concentration available to those of skill in the art.

Many preferred embodiments of the present invention provide foul-odor-mitigating compositions or devices comprising polymeric compounds. The presence of these polymeric compounds confer substantial benefits to many of the devices, systems and methods of the present invention, by significantly improving the stability of compositions and devices containing the OMRs. Not to be bound by theory, this effect is thought to be achieved either by the physical binding (such as intercalation) or by the chemical binding (i.e. covalent bonding) of the polymeric component of the invention with the other chemical components necessary for odor control. The polymeric compound may be a polymeric OMR, a polymeric promoter, a polymer solid support or other polymeric compound.

Binding of the OMR to a polymeric compound is particularly desirable where, as part of its intended use, the device is put in direct contact with liquids, especially aqueous liquids. The presence of the polymeric components of the present invention virtually eliminates the unintended, and undesirable, effect of OMR loss through the process of leaching. In particular, leaching of any supplemental non-polymeric agents, which might be added to bolster the odor-fighting effectiveness in a particular application, tends to be minimized. Exemplary embodiments devices that would benefit from this effect include, but are not limited to, landfill abatement covers, fabric, carpet, food packaging, pet litter, bags and containers for decomposing organic matter or animal waste and in medical applications such as colostomy bags, urine bags, body bags, bed pans and other containers for human waste or biological material.

This characteristic of rendering other compounds in the reagent package substantially not-leaching is also a substantial advantage in the formulation of odor-mitigating devices intended to contact the skin, hair or clothing of a user. In these contexts, it is frequently highly desirable that the migration of OMRs or other chemical components be effectively eliminated or significantly curtailed. Examples of embodiments of the present invention meant to come in contact with a user include, but are not limited to, disposable diapers, reusable diapers, foot-wear liners, wound dressings, compresses, sanitary or feminine hygiene pads, tampons, incontinence pads, flatulence pads, hair care caps or covers, odor-protecting face masks, deodorant pads, pet litter, clothing, footwear, upholstery fabric, carpeting and the like.

There are also other instances where, for different reasons, reagent migration would want to be avoided. For example, in odor-control devices where forced or passive air-flow could potentially facilitate odor-combatant reagent migration by mass transfer, it would be advantageous to polymerically bind the malodorant reactants in order to reduce reactant loss. This would increase both the efficacy and longevity of the device. This would be advantageous in the odor-mitigating devices mentioned above, but would also be advantageous in devices such as forced-air air filters or air fresheners, such as cabin air filters, unregulated-air air filters or air fresheners, and the like.

Regardless of whether or not the presence of polymeric OMR is sufficient to render the compounds within the device substantially non-leaching, the presence of polymeric compounds in the device yields additional benefits in the processing and manufacture of the odor-mitigating compositions or devices of the present invention. These benefits include lack of dusting and flaking of the composition or device, increased ease in the molding and shaping of the reagents into the appropriate conformations, as well as increased durability of the resultant device.

The addition of a polymeric component in the present invention can serve to render the OMR or promoters or other additives substantially non-leaching. Alternatively, the addition of one or more polymeric components to the odor-mitigating composition or device can assist in the processing or manufacturing of the composition or device, making those processes more facile and therefore less costly. It can also improve the durability of the device.

Air-borne malodorants or pollutants can be exposed to the compositions or devices of the present invention in several different ways. One application is to expose air-borne contaminants to compositions of the present invention by means of a forced-air apparatus. In certain embodiments of the present invention OMRs, with or without associated promoters or other additives are disposed on a support that is exposed to the contaminated air that is forced across or through the OMR-containing support. In certain embodiments, an impeller or fan of some type is used to move contaminated air through a reagent-containing filter. Typical examples of mechanical forced air embodiments of the present invention include interior air movers such kitchen hoods or bathroom fans; cabin air filters such as those found in automobiles, buses, trucks, trains boats and aircraft; and systems for removing malodorants or pollutants in industrial settings.

Another type of application for the present invention is in what the inventors call “matrix” systems. Applications of this type use large surface areas, and one or more layers of composition or device. The solution or gas containing the malodorant or contaminant can be brought in contact with the composition or device passively, by a pressure gradient, one or more fans, a blower system, or the like. Generally, such a system will be multi-dimensional. One example of such a system would be large volume baghouse of the type used in commercial textile mills or industrial facilities where coatings are applied. Other examples of possible applications for matrix-type systems include sewage treatment plants, waste processing facilities, or areas where large numbers of people gather such as schools, malls, enclosed stadiums, locker rooms, nurseries or large kitchens such as in the military or cafeterias. Essentially, these matrix systems are enlarged versions of the embodiments described above that can be used when large quantities of air need to be decontaminated.

A further application of the present invention are “stationary” type applications. Applications of this type involve airflow that is not forced into contact with the composition or device, but where the movement of contaminated air is through diffusion or some other type of unregulated air flow. Examples of such applications might include garbage pails, diaper pails or other enclosure that is outfitted with a porous composition or device. In such applications, the composition or device permits unregulated air flow between the exterior and interior of the enclosure, but prevents malodorants or contaminants from passing through. Alternate embodiments would include solid air fresheners.

Yet another embodiment of the present invention a “absorbent” type applications. In these applications the composition or device is brought in physical contact with a solid or liquid that contains the malodor or contaminants.

In certain embodiments the absorbent composition or device is intended to be brought in physical contact with the contaminated structure or substance and then subsequently removed. An example of such an embodiment is where a presently disclosed composition is disposed in a powder form, or adsorbed onto a powder support such as silica. Such powders could be used by applying them to carpeting, floors, upholstery, fabric or other surfaces. After allowing a sufficient time for odor elimination to take place, the powder may or may not be removed by a vacuum or similar device.

In other embodiments, the absorbent composition or device is placed in contact with a contaminated environment, but subsequent removal of the composition or device is not required. Examples of such embodiments would include diapers, garbage bags, body bags and the like. Typically such embodiments comprise an absorbent layer containing the OMRs.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Disposable Diaper Formulation

Taking the specific case of disposable diapers as a first example, the inventors have designed an efficacious formulation shown below. This product was shown to be effective in odor mitigation without adverse effect on diaper absorbency. Also, the formula shown is a “direct substitute” type. That is to say, it is intended to wholly replace the absorbent part of the diaper (absorbents are “built in” in this formula). This general formulation can also be used in applications that are similar to disposable diapers—such as incontinence pads or feminine hygiene pads. The general formula for the disposable diaper of the present embodiment is:
a) 92% Polymeric reagent.
b) 7% Promoter package.
c) 1% Non-polymeric reagent.
   The polymeric reagent portion breaks down as follows:
a) 82% HYSORB^{R} 8200 from BASF—or similar material (˜30% in acid form)
b) 9% Carbopol ULTREZ^{R} 10 from B.F. Goodrich
c) 9% “G” series polyacrylate from Grain Processing Corp. (˜20% in amide).
   Similarly, the promoter is as follows:
a) 30% 7-mole NI surfactant like ALFONIC^{R} 1412-60 or DUPONT L-588
b) 70% polyethylene glycol such as Dow's CHEMDELL^{R} 4000 to 8000.
   The non-polymeric reagent in this case is the disodium salt of dichloroisocyanuric acid, and is at a 1% level.
   Normalizing the numbers for 100% we have:
76% HYSORB^{R} 8200 from BASF—or similar material (˜30% in acid form)
8% Carbopol ULTREZ^{R} 10 from B.F. Goodrich
8% “G” series polyacrylate from Grain Processing Corp. (˜20% in amide)
2% 7-mole NI surfactant such as ALFONIC^{R} 1412-60 or DUPONT L-588 or similar nonionic surfactant from Shell (NEODOL^{R}) or Sasol.
5% polyethylene glycol such as Dow's CHEMDELL^{R} 4000 to 8000
1% disodium dichloroisocyanuric acid

In this case no pretreatment (or in-situ treatment either) of the polymeric reagent portion of the formula was done. The material of this composition can be directly substituted (generally a “one for one” on a weight basis) for the absorbent in practically any commercial disposable diaper product, to render a new product that is effective in combating diaper odors.

### EXAMPLE 2

### Footwear Inner Liner

For this and other examples that follow, the formulas are normalized to 100% (“synchronized” for direct substitution or application). For all applications, however, the stipulated attributes of this invention are maintained. That is to say, all contain polymeric reagent and modified polymeric promoter components that substantially reduce odor. Some have a small amount of non-polymeric reagent as well. For this particular application it should be noted that an adhesive has to be employed in order to secure the formula to the shoe interior. The composition was found to be effective in preventing odors:
70% HYSORB^{R} 8200 from BASF—or similar material (˜30% in acid form)
10% Carbopol ULTREZ^{R} 10 from B.F. Goodrich
10% “A 140” polyacrylate copolymer—Grain Processing Corp.
1% 7-mole NI surfactant like ALFONIC^{R} 1412-60 or DUPONT L-588 or similar
9% polyethylene glycol such as Dow's CHEMDELL^{R} 8000

### EXAMPLE 3

### Disposable Diaper (A More Economical Version)

Because cost considerations are always a major factor in arriving at commercial formulations, effective, but less costly versions of disposable diaper formulas can be fabricated. The following is one that has been shown to work:
80% HYSORB^{R} 8200 from BASF—or similar material (˜30% in acid form)
10% “G” series polyacrylate from Grain Processing Corp. (˜20% in amide)
1.5% 7-mole NI surfactant like ALFONIC^{R} 1412-60 or DUPONT L-588 or similar
7.5% polyethylene glycol such as Dow's CHEMDELL^{R} 8000
1% disodium dichloroisocyanuric acid

### EXAMPLE 4

### Disposable Diaper—Alternative Economical Version

A mixture of 75% ARIDALL^{R} 1460 or similar polyacrylate (˜35% in acid form), 13% A-120 graft copolymer (from Grain Processing Corp.), 1% 7-mole NI surfactant such as ALFONIC^{R} 1412-60 or DUPONT L-588 or similar, 10% polyethylene glycol such as Dow's CHEMDELL^{R} 8000 and 1% disodium dichloroisocyanuric acid was used in a disposable diaper for the control of malodors.

### EXAMPLE 5

### Disposable Diaper—Alternative Economical Version

A mixture of 79% HYSORB® 8200 from BASF—or similar material, 10% A-120 copolymer (from Grain Processing Corp.), 1% 7-mole NI surfactant such ALFONIC® 1412-60 or DUPONT L-588 or 10% polyethylene glycol such as Dow's CHEMDELL® 8000 was used in a disposable diaper for the control of malodors.

### EXAMPLE 6

### Landfill Odor Abatement Cover

The following are mixed:
37% FAVOR^{R} SXM 7500 polyacrylate
40% A-120 copolymer (from Grain Processing Corp.)
2% 7-mole NI surfactant such ALFONIC^{R} 1412-60 or DUPONT L-588 or
21% polyethylene glycol such as Dow's CHEMDELL^{R} 4000

In this application it can be necessary to repeat treatments on a periodic basis as shear volume of malodorous sources tend to increase in landfills over time. However, it was learned during this work that the same three general categories of malodorous molecules attributed to the other problem areas identified in this work, namely volatile acids, volatile bases (primarily amines) and neutral entities (primarily sulfur compounds like mercaptans) comprise 90+% of malodorous compounds emanating from landfills. Results to-date demonstrate that the polymeric OMR/polymeric promoter method of odor abatement set forth in this invention is successfully applied to control odors in this setting.

### EXAMPLE 7

### Composition for Porous Automotive Fresh Air Vent

As in example 2 (above), adhesive(s) (KODAK's photoadhesive and hydrolyzed PVA work nicely) can be employed to secure the odor-fighting composition (below) to vehicle fresh air vent filters. Filters can be manually breached with a fine needle or wire to ensure that adequate air flow is maintained after application of the adhesive and odor-combative composition (below). This embodiment works well in the present application as long as care is taken to ensure that there is substantial surface area for malodorant transformation, as well as adequate porosity for good air flow.
60% FAVOR^{R} SXM 7500 polyacrylate
10% Carbopol ULTREZ^{R} 10 from B.F. Goodrich
20% “A 140” polyacrylate copolymer—Grain Processing Corp.
1% 7-mole NI surfactant like ALFONIC^{R} 1412-60 or DUPONT L-588 or similar
9% polyethylene glycol such as Dow's CHEMDELL^{R} 8000

### EXAMPLE 8

### Polymeric Version for Restroom Application

The use of polymeric compounds in this formulation in a forced-air filtration device (such as those described in U.S. Pat. No. 6,528,014) has the advantage of improving the efficacy and longevity of the device. Not to be bound by theory, the improvement is believed to occur because important chemical components are not lost due to mass transfer effects that normally occur during operation of the device.
30% “G” series polyacrylate from Grain Processing Corp. (˜20% in amide)
2% disodium dichloroisocyanuric acid
22% FAVOR^{R} SXM 7500 polyacrylate
10% Carbopol ULTREZ^{R} 10 from B.F. Goodrich
20% “A 140” polyacrylate copolymer—Grain Processing Corp.
3% 7-mole NI surfactant like ALFONIC^{R} 1412-60 or DUPONT L-588 or similar 13% polyethylene glycol such as Dow's CHEMDELL^{R} 8000

### EXAMPLE 9

### Feminine Hygiene Application

This use is similar in demand and product configuration to the disposable diaper. This following formulation will be effective in preventing odors in feminine pads:
68% FAVOR^{R} SXM 7500 from Stockhausen (30% acid form˜)
9% Carbopol ULTREZ^{R} 10 from B.F. Goodrich
10% “G” series polyacrylate from Grain Processing Corp. (˜20% in amide)
2% 7-mole NI surfactant such as ALFONIC^{R} 1412-60 or DUPONT L-588 or similar
10% polyethylene glycol such as Dow's CHEMDELL^{R} 8000
1% disodium dichloroisocyanuric acid

### EXAMPLE 10

### Hair Care Cap or Cover

As in two previous examples cited above, adhesive can be employed to secure the polymeric composition of this invention to a hair covering that is used during the application of hair permanents or dying. The principal malodor here is ammonia. The following formulation is effective in controlling odors from hair care products:
69% HYSORB^{R} 8200 from BASF—or similar material (˜30% in acid form)
20% “G” series polyacrylate from Grain Processing Corp. (˜20% in amide)
1.5% 7-mole NI surfactant like ALFONIC^{R} 1412-60 or DUPONT L-588 or similar
7.5% polyethylene glycol such as Dow's CHEMDELL^{R} 8000
2% of a 50/50 mixture of disodium dichloroisocyanuric acid and citric acid

### EXAMPLE 11

### Incontinence Pads

As in the case of feminine hygiene products and disposable diapers, incontinence pads incorporating the innovations of this compositional invention address very similar malodor problems. As reflected in the following demonstrably efficacious formulation, incontinence is efficiently ameliorated by the same kind of polymeric reagent/polymeric promoter product as was successfully applied to disposable diapers and the analogous applications previously discussed. The following formulation is useful in incontinence pads:
19% “G” series polyacrylate from Grain Processing Corp. (˜20% in amide)
70% HYSORB^{R} 8200 from BASF—or similar material (˜30% in acid form)
9% A-120 graft copolymer (from Grain Processing Corp.)
1% 7-mole NI surfactant such as ALFONIC^{R} 1412-60 or DUPONT L-588 or similar (like
Shell Chemical's NEODOL^{R} nonionic surfactant series)
1% disodium dichloroisocyanuric acid

### EXAMPLE 12

### Disposable Diaper—General

The following formulation is effective as part of a diaper for the control of malodors:
75% HYSORB^{R} 8200 from BASF—or similar material (˜30% in acid form)
13% A-120 graft copolymer (from Grain Processing Corp.)
1% 7-mole NI surfactant such as ALFONIC^{R} 1412-60 or DUPONT L-588 or similar
10% polyethylene glycol such as Dow's CHEMDELL^{R} 4000 to 8000
1% disodium dichloroisocyanuric acid

### EXAMPLE 13

### Disposable Diaper—Urine Emphasis

The following formulation is effective as part of a diaper for the control of malodors associated with urine:
10% Carbopol ULTREZ^{R} 10 from B.F. Goodrich
72% HYSORB^{R} 8200 from BASF—or similar material
10% A-120 copolymer (from Grain Processing Corp.)
1% 7-mole NI surfactant such ALFONIC^{R} 1412-60 or DUPONT L-588 or
7% polyethylene glycol such as Dow's CHEMDELL^{R} 8000

### EXAMPLE 14

### Hair Care Cap, or Cover

The following were mixed and used as part of a hair care cap or cover for the control of malodors:
73% HYSORB^{R} 8200 from BASF—or similar material (˜30% in acid form)
16% A-140 polyacrylate graft copolymer (˜20% in amide)
1.5% 7-mole NI surfactant like ALFONIC^{R} 1412-60 or DUPONT L-588 or similar
7.5% polyethylene glycol such as Dow's CHEMDELL^{R} 8000
2% of a 50/50 mixture of disodium dichloroisocyanuric acid and citric acid

### EXAMPLE 15

### Hair Care Cap, or Cover

The following were mixed and used as part of a diaper for the control of malodors and contaminants associated with hair care products:
55% DUPONT L-588 nonionic surfactant
15% Carbopol ULTREZ^{R} 10 from B.F. Goodrich
30% citric acid.
   This material in this embodiment is applied in liquid form to the net or cover. In this case there is a limited time to do it as it sets up.

### EXAMPLE 16

### Diaper Application—Fecal

The following were mixed and used as part of a diaper for the control of malodors associated with feces:
15% DUPONT L-588 nonionic surfactant
4% Carbopol ULTREZ^{R} 10 from B.F. Goodrich
75% Potassium carbonate
6% dichloroisocyanuric acid—sodium salt
   This mixture is applied in a powder form to the diaper super-absorbent material in the diaper. Thus, the functional formula could be said to contain a polyacrylate such as HYSORB^{R} 8200 though it is not listed above.

### EXAMPLE 17

### Forced-Air Filtration Systems

For a bathroom odor application a solid state device was conceived and built which housed the material for malodorant conversion on a porous fabric. The configuration and operational limitations of the device necessitated that the rate at which transformation occurred be as high as possible. Additional practical considerations highlighted the importance of maintaining a “quasi-solid” fabric so the inconveniences associated with liquids could be avoided. An inert inorganic support was frequently employed to increase surface area and the concomitant elevated number of sites for transformations to occur. Use of these supports did not have a noticeable negative impact on the longevity of the composition fabric. Indeed, they too improved overall performance of the device. In any case, test results with the bathroom-odor-removal device showed that promoters, with or without supports, significantly enhanced odor mitigation performance over cases where reagents alone were used.

### EXAMPLE 18

### Safely Reducing Urine Odors in Diapers

Sodium dichloroisocyanurate (0.01 to 10% formula weight) combined with a polyacylate superabsorbent polymer (20 to 90% formula weight) is effective for the control of malodors. The composition is combined used in devices such as diapers, feminine pads and incontinence pads or wherever it is desirable to neutralize malodorants associated with urine. A preferred embodiment, consisting of 2.5% sodium dichloroisocyanurate and 97.50% polyacrylate polymer (superabsorbent) has been shown to be effective in safely reducing odors in diapers.

### EXAMPLE 19

### Safely Reducing Urine Odors in Diapers

A composition is prepared comprising 0.01-10% sodium dichloroisocyanurate, 10-90% polyacrylate polymer (superabsorbent), 0-70% citric acid, 0.01-20% ethoxylate such as DuPont L-588 ethyoxylate, and 0.1-10% fumed silica. The mixture is used as part of a device such as a diaper, feminine pad or incontinence pad where it is desirable to neutralize malodorants associated with urine. A preferred embodiment composed of 2.0% sodium dichloroisocyanurate, 38.5% polyacrylate polymer (superabsorbent), 53% citric acid, 3.5% ethoxylate, and 3.0% fumed silica has been shown to be effective in safely reducing odors in diapers.

### EXAMPLE 20

### Safely Reducing Urine Odor in Diapers

A composition is prepared comprising 0.01-10% sodium dichloroisocyanurate, 10-90% polyacrylate polymer (superabsorbent), 0-70% citric acid, and 0.01-20% polyethylene glycol. The mixture is used as part of a device such as a diaper, feminine pad or incontinence pad where it is desirable to neutralize malodorants associated with urine. A preferred embodiment composed of 1.75% sodium dichloroisocyanurate, 41.25% polyacrylate polymer (superabsorbent), 52.0% citric acid, and 5.0% polyethylene glycol has been shown to be effective in safely reducing odors in diapers.

### EXAMPLE 21

### Safely Reducing Urine Odor in Diapers

A composition is prepared comprising 10-90% polyacylate polymer (superabsorbent), 0-70% citric acid, 0.01-20% ethoxylate such as DuPont L-588 ethyoxylate, and 0.1-10% fumed silica. The mixture is used as part of a device such as a diaper, feminine pad or incontinence pad where it is desirable to neutralize malodorants associated with urine. A preferred embodiment composed of 38.0% polyacylate polymer (superabsorbent), 50.0% citric acid, 1.0% ethoxylate such as DuPont L-588 ethyoxylate, and 1.0% fumed silica (as a solid support) has been shown to be effective in safely reducing odors in diapers.

### EXAMPLE 22

### Safely Reducing Fecal Odors in Diapers

A composition is prepared comprising 0.01-10% sodium dichloroisocyanurate, 10-90% polyacrylate polymer (superabsorbent), 0-70% potassium carbonate, 0.01-20% ethoxylate such as DuPont L-588 ethyoxylate, and 0.1-10% fumed silica (as a solid support). The mixture is used as part of a device such as a diaper or incontinence pad where it is desirable to neutralize malodorants associated with feces. A preferred embodiment composed of 5.5% sodium dichloroisocyanurate, 46% polyacrylate polymer (superabsorbent), 45% potassium carbonate, 2.0% ethoxylate such as DuPont L-588 ethyoxylate, and 1.5% fumed silica (as a solid support) has been shown to be highly effective in reducing fecal odors in diapers.

### EXAMPLE 22

### Safely Reducing Fecal Odors in Diapers

A composition is prepared comprising 0.01-10% sodium dichloroisocyanuraie, 10-90% polyacrylate polymer (superabsorbent) and 0-70% potassium carbonate. The mixture is used as part of a device such as a diaper or incontinence pad where it is desirable to neutralize malodorants associated with feces. A preferred embodiment composed of 6.0% sodium dichloroisocyanurate, 44% polyacrylate polymer (superabsorbent) and 50% potassium carbonate, has been shown to be effective in reducing fecal odors in diapers.

### EXAMPLE 23

### Hair Care Cap, or Cover

A mixture of 1-75% citric acid, 1-75% Ultrez 10 polyacrylate (B.F.Goodrich) and 10-65% ethoxylate such as DuPont L-588 ethyoxylate is prepared and applied to a device such as permanent hair color cap for the control of malodors and other contaminants associated with certain hair care products. A preferred embodiment of 25% citric acid, 20% Ultrez 10 polyacrylate and 55% ethyoxylate was prepared and applied to a hair care cap. The mixture forms an elastic pseudopolymer after curing for a short period of time. The compound was found not to leach from the cap and to be effective in the control of odors.

### EXAMPLE 24

### Safely Reducing Urine Odor in Diapers

A composition is prepared comprising 0.01-10% sodium sulfite, 10-90% polyacrylate polymer (superabsorbent) and 0.01-10% potassium carbonate. The mixture is used as part of a device such as a diaper, feminine pad or incontinence pad where it is desirable to neutralize malodorants associated with urine. A preferred embodiment composed of 6.0% sodium sulfite, 88% polyacrylate polymer (superabsorbent) and 6.0% potassium carbonate will be effective in controlling urine odors. In this example, it is a reducing agent, sodium sulfite, that acts to neutralize malodorants.

### EXAMPLE 25

### Diaper Fecal Odor Mitigation

The following mixture was found to be effective in mitigating diaper odors:
45.9% potassium carbonate (monomer reagent)
0.1% sodium dichloroisocyanurate (monomer reagent)
50.0% polyacrylate superabsorbent like FAVOR SXM 880 (polymer reagent)
1.5% fumed silica (support)
2.5% 8 mole nonionic ethoxylate like DuPont L588 (monomer promoter)

### EXAMPLE 26

### Diaper Urine Odor Example

The following mixture will be useful in the prevention of diaper urine odors:
2.0% meta-chloroperbenzoic acid (non-polymeric OMR)
98.0& polyacrylate superabsorbent (such as FAVOR SXM 880)

### EXAMPLE 27

### Diaper Urine Odor

The following mixture will be useful in the prevention of diaper urine odors:
2.0% meta-chloroperbenzoic acid (non-polymeric OMR)
2.0% 8 mole ethoxylate like DuPont L588
96% polyacrylate superabsorbent (such as FAVOR SXM 880)

### EXAMPLE 28

### Diaper Urine Odor

The following mixture will be useful in the prevention of diaper urine odors:
2.0% perbenzoic acid (non-polymeric OMR)
98.0% polyacrylate superabsorbent (such as FAVOR SXM 880)

All of the compositions and methods disclosed and claimed herein can be made and executed without undue experimentation in light of the present disclosure. While the compositions and methods of this invention have been described in terms of preferred embodiments, it will be apparent to those of skill in the art that variations can be applied to the compositions and/or methods and in the steps or in the sequence of steps of the methods described herein without departing from the concept, spirit and scope of the invention. More specifically, it will be apparent that certain agents that are chemically or physiologically related can be substituted for the agents described herein while the same or similar results would be achieved. All such similar substitutes and modifications apparent to those skilled in the art are deemed to be within the spirit, scope and concept of the invention as defined by the appended claims.

1. An odor-mitigating composition comprising a polymeric odor-mitigating reagent having one or more functional groups selected the group consisting of a Lewis Acid, a Lewis Base, an oxidizing agent, a hydro-sulfur labile compound or a reducing agent, wherein the polymeric reagent is substantially non-leaching and wherein the polymeric odor-mitigating reagent is contacted to a solid support.

2. The composition of claim 1 disposed in a device for use in contact with skin, hair or clothing of a user.

3. The composition of claim 2 wherein the device is a diaper.

4. The composition of claim 2 wherein the device is a shoe liner.

5. The composition of claim 2 wherein the device is a wound dressing.

6. The composition of claim 2 wherein the device is a sanitary pad.

7. The composition of claim 2 wherein in the device is a face mask.

8. The composition of claim 2 wherein the device is a hair care cap or cover.

9. The composition of claim 1, further comprising one or more promoters.

10. The composition of claim 1, further comprising a non-polymeric odor-mitigating reagent.

11. The composition of claim 10 wherein the non-polymeric odor-mitigating reagent is a Lewis Acid.

12. The composition of claim 10 wherein the non-polymeric odor-mitigating reagent is a Lewis Base.

13. The composition of claim 10 wherein the non-polymeric reagent is an oxidizing agent or hydro-sulfur labile compound.

14. The composition of claim 13 wherein the oxidizing agent is a sodium salt of dichloroisocyanuric acid.

15. An odor-mitigating composition comprising an admixture of:
(a) a polymeric promoter,
(b) an OMR, wherein the OMR is substantially non-leaching in the presence of the polymeric promoter, wherein the OMR is contacted to a solid support.

16. The composition of claim 15 wherein the OMR comprises a Lewis Acid.

17. The composition of claim 15 wherein the OMR comprises a Lewis Base.

18. The composition of claim 15 wherein the OMR comprises an oxidizing agent or a hydro-sulfur labile compound.

19. The composition of claim 15 wherein the OMR comprises 0.1%-10.0% by weight of oxidizing agent.

20. The composition of claim 15 wherein the OMR is the sodium salt of dichloroisocyanuric acid.

21. The composition of claim 15 wherein the OMR is polymeric.

22. The composition of claim 15 wherein the polymeric promoter comprises a polyether material.

23. The composition of claim 22 wherein the polyether material is polypropylene oxide, polyethylene oxide or carbowax.

24. The composition of claim 15 further comprising a low molecular weight polyether.

25. The composition of claim 24 wherein the low molecular weight polyether is a non-ionic surfactant ethylene oxide adduct having 6 or more ethylene oxide units.

26. The composition of claim 15 further comprising a disinfectant.

27. The composition of claim 26 wherein the disinfectant comprises ethanol or a quarternary ammonium halide.

28. A device containing the composition of claim 15, wherein the device contains layered or segregated composites, and wherein one or more of the composites comprises an OMR comprising a Lewis Acid, and one or more of the remaining composites comprises an OMR comprising a Lewis Base.

29. The device of claim 28 further comprising a oxidizing agent or a hydro-sulfur labile compound as an OMR in one or more of the composites.

30. A device comprising the composition of claim 15.

31. A diaper comprising the composition of claim 15.

32. A shoe liner comprising the composition of claim 15.

33. A wound dressing comprising the composition of claim 15.

34. A sanitary pad comprising the composition of claim 15.

35. A face mask comprising the composition of claim 15.

36. A hair care cap or cover comprising the composition of claim 15.

37. Use of the device of claim 30 outdoors.

38. An odor-mitigating device comprising:
(a) an effective amount of a bleach or chlorine donor, and
(b) a polymer capable of neutralizing hydrochloric acid.

39. The device of claim 38, wherein the polymer comprises carboxylate salt functional groups.

40. The device of claim 39, wherein the polymer is a super-absorbent polymer.

41. The device of claim 38 wherein the device is a disposable diaper.

42. The device of claim 38 wherein the device is an incontinence pad.

43. A method of mitigating a malodorant or other contaminant comprising exposing the composition of claim 15 to an environment containing the malodorant or other contaminant.

44. The method of claim 43 wherein the malodorant or other contaminant is air-borne.

45. The method of claim 43 wherein the malodorant or other contaminant is in liquid solution.

46. The method of claim 43, wherein the malodorant is ammonia.
